(19) United States
(12) Patent Application Publication
(10) Pub. No.: US 2016/0199021 A1
(43) Pub. Date: Jul. 14, 2016

(54) DISPOSABLE STETHOSCOPE COVER

(71) Applicant: Sarah D. Dula, Lenoir, NC (US)

(72) Inventor: Sarah D. Dula, Lenoir, NC (US)

(21) Appl. No.: 14/593,020

(22) Filed: Jan. 9, 2015

(51) Int. Cl. A61B 7/02; A61B 19/08

(52) U.S. Cl. CPC A61B 7/02; A61B 19/081

(57) ABSTRACT

A stethoscope cover including: a disc shaped body; an opening within the body; and an elastic band, where the elastic band surrounds the opening to secure the cover over a chest piece of a stethoscope. Preferably, the body includes a flexible material for an easy fit over the chest piece. Further preferably, the cover may be disposable as a single use item.

### BACKGROUND OF THE INVENTION

1. Field of Invention

The present invention relates to a disposable stethoscope cover for use in a medical examination room.

2. Description of Related Art

Stethoscopes are a common medical tool that is used by doctors and nurses to examine patients. The stethoscope provides a device for a medical professional to listen to the internal sounds of a human body. Typically it is used to listen to the lungs, heart and blood flow in the arteries and veins. The stethoscopes were also commonly used when checking an individual's blood pressure. The stethoscope includes earpieces that are placed into a medical professional's ear and a chest piece that is placed directly against a patient's skin in order to listen to the internal sounds. On the opposite side of the chest piece typically is a bell that is held by the physician. The stethoscope may be used multiple times throughout a regular business day of a medical professional. Normally the chest piece remains exposed and comes into direct contact with a patient's skin. Presently there doesn't exist any particular means to cover this chest piece or diaphragm of the stethoscope during use. It would be advantageous to have a cover that may cover the chest piece of the stethoscope and therefore reduce the possibility of the transfer of germs and bacteria while using a stethoscope.

### SUMMARY OF THE INVENTION

The present invention relates to a stethoscope cover comprising: a disc shaped body; an opening within the body; and an elastic band, where the elastic band surrounds the opening to secure the cover over a chest piece of a stethoscope. Preferably, the body includes a flexible material for an easy fit over the chest piece. Further preferably, the cover may be disposable as a single use item.

### BRIEF DESCRIPTION OF DRAWING

FIG. 1 depicts a disposable stethoscope cover in accordance with the present invention.

### DETAILED DESCRIPTION

The present invention relates to a sterile cover that is used to cover the chest piece or diaphragm of a stethoscope. The cover of the present invention provides a sanitary material that covers the chest piece of the stethoscope. A small opening is provided within the cover that allows the cover to slip over the stethoscope chest piece. The opening includes an elastic circumference that provides for a secure grip of the cover over the stethoscope during use. Ideally the present invention is used on a patient and then discarded once the examination is complete.

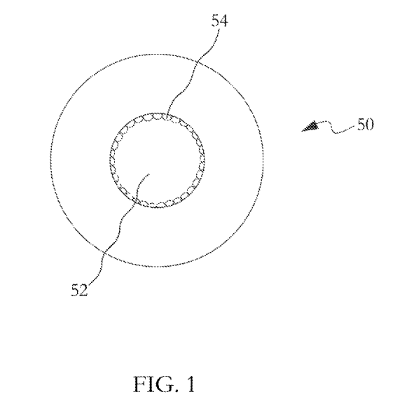

FIG. 1 depicts a stethoscope cover 50 in accordance with the present invention. As shown the stethoscope cover is substantially disc type shape although flexible. The stethoscope cover 50 includes an opening 52 that allows for the insertion of a chest piece or diaphragm of a stethoscope. Surrounding the opening 52 is an elastic band 54 that provides a secure gripping means for the cover once placed over the chest piece. Use of the stethoscope cover accordance to the present invention reduces the transferred germs and bacteria between patients. The cover also protects the chest piece and eliminates accumulation of dirt or debris within the chest piece during use. A medical professional slides the cover over the chest piece during use and discards of the cover after one use to avoid transmission of germs or bacteria. The instant invention has been shown and described in what it considers to be the most practical and preferred embodiments. It is recognized, however, that departures may be made there from within the scope of the invention and that obvious modifications will occur to a person skilled in the art.

1. A stethoscope cover comprising:
a. a disc shaped body, adapted to cover an entire bell of a stethoscope;
b. an opening within the body; and
c. an elastic band, where the elastic band surrounds the opening to secure the cover over a chest piece of a stethoscope.

2. The stethoscope cover according to claim 1, where the body includes a flexible material.

3. The stethoscope cover according to claim 1, where the cover is disposable.

4. (canceled)
